(19) United States
(12) Patent Application Publication
(10) Pub. No.: US 2022/0117507 A1
(43) Pub. Date: Apr. 21, 2022

(54) SYSTEM AND METHOD FOR PROCESSING MEASUREMENT DATA FROM ELECTROCARDIOGRAM ELECTRODES

(71) Applicant: Venster Medical, Inc, Menlo Park, CA (US)

(72) Inventors: Peter M. VAN DAM, Arnhem (NL); David PICKHAM, Menlo Park, CA (US)

(21) Appl. No.: 17/072,533

(22) Filed: Oct. 16, 2020

(51) Int. Cl. A61B 5/0428; A61B 5/0408; A61B 5/044; A61B 5/00; A61B 5/06; A61B 8/00; A61B 8/02

(52) U.S. Cl. CPC A61B 5/0428; A61B 5/0408; A61B 5/044; A61B 5/6823; A61B 5/066; A61B 8/4444; A61B 5/0073; A61B 5/7292; A61B 8/56; A61B 8/02; A61B 5/0077

(57) ABSTRACT

A system and method for processing measurement data from ECG electrodes. The method includes obtaining a three-dimensional image of the torso of the subject including position information of the electrodes; obtaining ultrasound data of the heart of the subject; and modifying a non-patient-specific three-dimensional anatomical model into a patient-specific three-dimensional model of the heart and torso of the subject on the basis of the ultrasound data. The method includes using electrocardiogram data and the three dimensional patient-specific anatomical model for estimating the distribution, fluctuation and/or movement of electrical activity through heart tissue.

### FIELD OF THE INVENTION

The present invention relates to electrocardiography, in particular to an electrocardiography device. Also, the present invention relates to inverse electrocardiography, such as determining electrical activity of heart tissue. More in particular the invention relates to determining a patient-specific anatomical model of the heart for use in determining electrical activation of heart tissue.

### BACKGROUND TO THE INVENTION

Electrocardiogram, ECG, measurements have been used for over a hundred years for obtaining insight in the functioning, and malfunctioning, of the heart of subjects. In recent years inverse electrocardiography has increasingly been used for the same purpose, providing marked advantages over classic electrocardiography. The inverse problem of electrocardiography consists in reconstructing cardiac electrical activity from given body surface electrocardiographic measurements. The inventors have to date made progress in so called inverse computations where e.g. an activation sequence and/or other parameters of the heart are estimated from surface electrocardiograms. Inverse imaging of electrical activity of a heart muscle is for instance described in published patent application US-2012-0157822-A1. The method generally relies on processing measurement data from electrocardiogram, ECG, electrodes on a subject. Typically the ECG measurements are obtained not directly on the myocardium but on the intact skin of the subject. The modeling of the electrical activity of the heart requires a model of the heart and torso, where the heart represents the source of the ECG signals and the torso the surface where ECG signals measured. A three-dimensional, 3D, anatomical model of the heart and torso of the subject is used to correlate ECG electrode locations on the skin to the position of the heart inside the torso. The inverse electrocardiography ultimately results in a 3D model of the heart of the subject displaying, e.g. to a practitioner, the electrical activity of the heart, such as location(s) of activation of heart depolarization, fluctuation and/or movement of electrical activity through heart tissue, or the like. The 3D model of electrical heart activity provides useful information to the practitioner. Deviations and/or anomalies in the detected electrical activity of the heart can point to certain defects or diseases.

In WO2015/170978A1 the inventors describe a computer implemented method for processing measurement data from ECG electrodes on a subject including the computer obtaining a 3D anatomical model of the torso of the subject, obtaining a 3D image of the torso of the subject including position information of the electrodes, aligning the 3D image and the 3D model, determining a position of each electrode in the 3D from the 3D image; and using the positions of the electrodes in the 3D model for estimating electrical hear activity, such as activation, distribution, fluctuation and/or movement of electrical activity through heart tissue.

Traditionally the 3D anatomical model of the heart and torso for inverse electrocardiography is created from magnetic resonance imaging (MRI) or computed tomography (CT) images providing a full 3D or quasi-3D (e.g. a full stack of 2D slices) medical image of the heart and torso. This however, is an expensive and cumbersome procedure and not always available to health care experts, whereas the recording of the ECG is simple and readily available within the health care system.

### SUMMARY OF THE INVENTION

It is an object to generate a 3D anatomical model of the heart and torso for use in determining electrical activity of heart tissue in a less expensive and less cumbersome way. It is an object to provide an improved method and system for determining electrical activation of heart tissue.

According to an aspect is provided an electrocardiogram, ECG, device. The ECG device comprises one or more electrodes arranged to be placed on a subject. The ECG device comprises one or more ultrasound probes. The ECG device comprises a three-dimensional, 3D, camera. The ECG device comprises a processor. The processor is configured to obtain, from the 3D camera, a 3D image of the torso of the subject including position information of the electrodes on the torso of the subject. The processor is configured to obtain, e.g. from a database, a non-patient-specific 3D anatomical model of the heart and torso for the subject. The non-patient specific 3D anatomical model can be selected on the basis of the 3D image. The processor is configured to obtain, from the one or more ultrasound probes, ultrasound data of the heart of the subject. The processor may be configured to obtain position information of the one or more ultrasound probes on the basis of the 3D image. The processor is configured to modify the non-patient-specific 3D anatomical model into a patient-specific 3D model of the heart and torso of the subject on the basis of the ultrasound data. The processor is configured to determine a position of each electrode in the patient-specific 3D anatomical model based on the 3D image. The processor is configured to obtain electrocardiogram data from the electrodes. The processor is configured to use the electrocardiogram data and the positions of the electrodes in the 3D patient-specific anatomical model for determining a 3D model of electrical heart activity for the subject.

Hence, the ECG device is arranged to perform inverse electrocardiography. The ECG device can include display means, such as a display screen or printer, for displaying the 3D model of electrical heart activity to a user. The 3D model of electrical heart activity can be displayed as a, e.g. rotatable and/or movable and or scalable, 2D rendering on the display means. The 3D model of electrical heart activity can e.g. have a value representative of the electrical heart activity associated with each location, such as each node, on the surface of the 3D model of the heart. The values can e.g. represent electrical activation sequence, distribution, fluctuation and/or movement of electrical activity through heart tissue, heart synchronicity, or the like. The 3D model of electrical heart activity can be represented in false colors on the surface of the 3D model of the heart. Each value can e.g. be associated with a particular color. The 3D model of electrical heart activity can e.g. be represented in contours of equal value on the surface of the patient-specific 3D model of the heart.

According to an aspect is provided a method, such as a computer implemented method, for processing measurement data from electrocardiogram, ECG, electrodes on a subject. The method includes obtaining a three-dimensional, 3D, image of the torso of the subject including position information of the electrodes, e.g. using a 3D camera. The method includes obtaining, e.g. selecting from a database, a non-patient-specific 3D anatomical model of the heart and torso for the subject on the basis of the 3D image. Alternatively, or additionally, the non-patient-specific 3D anatomical model can e.g. be obtained from parameters derived from the 3D image. Alternatively, or additionally, the non-patient-specific 3D anatomical model can e.g. be obtained from a machine learning device on the basis of the 3D image. The method includes obtaining ultrasound data of the heart of the subject and modifying the non-patient-specific three-dimensional anatomical model into a patient-specific three-dimensional model of the heart and torso of the subject on the basis of the ultrasound data. The method includes determining a position of each electrode in the patient-specific three-dimensional anatomical model based on the three-dimensional image. The method includes obtaining electrocardiogram data from the electrodes and using the electrocardiogram data and the positions of the electrodes in the three-dimensional patient-specific anatomical model for estimating the distribution, fluctuation and/or movement of electrical activity through heart tissue.

The ultrasound imaging technique is readily available to practitioners. The ultrasound imaging requires a probe positioned on the skin of a patient. However, no bulky and expensive equipment such as MRI or CT apparatus are required. Based on acquired ultrasound data the contours of the blood cavities and myocardial tissue structures, such a papillary muscles or valves, can be identified. The ultrasound images provide a 3D image of, a portion of, the heart or 2D cross sections of the heart. The ultrasound data can thus be used to verify whether or not the 3D heart model in the non-patient-specific 3D anatomical model corresponds to the heart of the subject. The ultrasound data can be used to modify the non-patient-specific three-dimensional anatomical model into a patient-specific three-dimensional model of the heart and torso of the subject. Thereto the shape, dimensions, position and orientation of the heart in the non-patient-specific 3D anatomical model can be altered to correspond to the heart of the subject, thus obtaining the user-specific 3D anatomical model of the heart and torso.

The obtaining of ultrasound data can include obtaining ultrasound images from a plurality of locations and/or angles on the torso, e.g. using a plurality of ultrasound probes.

To be able to determine the shape, dimensions, position and orientation of the heart relative to the torso from the ultrasound data the exact position and orientation of the ultrasound beam on the torso needs to be known. The method can include determining a position and/or orientation of the ultrasound probe(s) from the 3D image. It is also possible that the position and/or orientation of the ultrasound probe(s) is determined by the computer on the basis of position signals of the probe(s). It is also possible that the position and/or orientation of the ultrasound probe(s) is inputted into the computer, e.g. manually. If the position and orientation of the ultrasound probe(s) has been determined the ultrasound images of the respective probe(s) can be aligned and/or registered with the non-patient-specific 3D anatomical model. The ultrasound images and the 3D image can be put in the same orthogonal coordinate system. Once all images are put in the same coordinate system, the ultrasound images can be used to reconstruct a part of the heart captured by the ultrasound images. The patient-specific 3D anatomical model of the heart and torso can be constructed on the basis of the ultrasound images.

Optionally at least one of the electrodes includes an ultrasound probe. Thus a combined ECG/ultrasound probe is provided capable of measuring the ECG as well as to send and retrieve ultrasound data from which ultrasound images can be obtained. The position and/or direction of the combined probe can be determined, e.g. from the 3D image. As the skin surface can be accurately determined by a 3D image obtained with a 3D camera, as well as any object put on the skin, position and orientation of the combined ECG electrodes/ultrasound probe can be accurately determined. It will be appreciated that similarly the position and orientation of ultrasound probes (separate from ECG electrodes) on the skin can easily be determined.

Optionally, the obtaining of the ultrasound data is synchronized to a heart rhythm obtained from the electrocardiogram data. As the obtaining of the ultrasound image is triggered to the ECG, the heart contours can be determined at the onset QRS, i.e. in the end-diastolic phase or any intermediate contractions/relaxation phase of the heart. Optionally, the breathing of the patient, which influences the orientation of the heart within the thorax, can be taken into account.

The method can include determining whether or not the obtained ultrasound data is sufficient for modifying the non-patient-specific three-dimensional anatomical model into a patient-specific three-dimensional model of the heart and torso, and if the obtained ultrasound data is insufficient obtaining additional ultrasound data of the heart of the subject. The additional ultrasound data can be obtained from an additional location and/or additional angle on the torso. The method can include determining a desired position for the additional location and/or a desired orientation for the additional angle. The obtaining of additional ultrasound data can be repeated until the data is sufficient for modifying the non-patient-specific three-dimensional anatomical model into a patient-specific three-dimensional model of the heart and torso.

The data can be sufficient if a large part of the heart surface is captured with the ultrasound probes. For instance when the portion of heart surface data captured with ultrasound exceeds a threshold value, e.g. 70%, the remainder of the heart surface can be estimated. Or for instance when just an endocardium is captured and only a part of the epicardium, a constant wall thickness can be assumed to estimate the epicardial surface based on endocardial segmentation points.

Optionally, the method includes estimating the position of heart scar tissue on the basis of absence or limited motion of the heart wall in the ultrasound data. It is appreciated that a non-moving heart wall part is not exactly the same as a scar in the heart tissue. Nevertheless, it has been found that a part of the heart wall which display no or very limited movement in practice provides a good initial estimate for the presence of scar tissue.

Optionally, the obtaining of the non-patient-specific 3D anatomical model of the heart and torso includes, obtaining the non-patient-specific 3D anatomical model of the heart and torso on the basis of thorax contours determined from the 3D image. Additionally, or alternatively, the obtaining of the non-patient-specific 3D anatomical model of the heart and torso includes, obtaining a non-patient-specific three-dimensional anatomical model of the heart and torso on the basis of at least one of gender, age, weight, body length, chest circumference, frame size, and body-mass-index.

Optionally, the non-patient-specific 3D anatomical model of the torso for the subject is determined by selection from a database. Thereto can be provided a database including a plurality of 3D anatomical models of torsos. The 3D models can include geometries of torsos including the heart and, optionally, including geometries of one or more of lungs, blood cavities, ribcage, fat and any other relevant tissue in the torso. The non-patient-specific 3D anatomical models are mutually different. The 3D anatomical models may represent different possible subjects. The 3D anatomical models may e.g. be representative of subjects of different gender, age, weight, body length, chest circumference, frame size, body-mass-index (BMI), etc. The 3D anatomical models may also differ in view of medical criteria, such as blood pressure. It will be appreciated that each 3D anatomical model in the database can e.g. be derived from a medical imaging modality, such as MRI, CT, PET-CT, ultrasound, or the like, from a respective reference subject. It is also possible that some or all 3D anatomical models in the database are fictitious renderings of fictitious reference subjects.

The method can then include selecting, from the plurality of non-patient-specific 3D anatomical models in the database, the 3D anatomical model showing closest conformity to the torso of the subject. The selection may be made on the basis of parameters, such as gender, age, weight, body length, chest circumference, frame size, BMI, etc. Such selection may be automated on the basis of parameters of the subject that are already known, e.g. from measurements, questions or tests. From the 3D image several measurements can be computed, e.g. chest circumference, height of the torso etc. These measurements can be used in selecting the appropriate 3D model from the database.

The selection may also be based on visual comparison of the 3D image of the torso of the subject with the 3D models in the database. Such selection may be automated on the basis of pattern recognition. Optionally, the method includes, after selecting a non-patient-specific 3D anatomical model from the database, scaling the 3D anatomical model to the 3D image of the torso of the subject, and/or scaling the 3D image to the 3D anatomical model. This enhances conformity of the non-patient-specific 3D anatomical model to the 3D image. The non-patient-specific 3D anatomical model can be scaled so as to have the outer surface of the non-patient-specific 3D anatomical model correspond with the outer surface of the torso of the subject as obtained from the 3D image. When the non-patient-specific 3D anatomical model is scaled, also dimensions and positions of internal structures such as the heart can be scaled.

It is also possible to take parameters of the subject into account when scaling the non-patient-specific 3D anatomical model. For example, the scaling can be dependent on the amount of body fat and frame size of the subject. In a subject with more body fat, the chest circumference can be larger in relation to the dimensions of heart, and e.g. lungs, than in a subject with less body fat.

Optionally, the method includes placing a marker on the torso of the subject, for example at the xyphoid. The marker is arranged to be identifiable in the 3D image of the torso of the subject. The marker can be used for determining the position of the heart. The marker at the xyphoid can be used as a reference for the lower end of the heart.

It is also possible to take parameters of the subject into account when determining a position of the heart within the 3D anatomical model. Such parameter can e.g. be weight or age of the subject. The weight can be indicative of a large abdomen, which pushes the heart upwards. Therefore, a vertical position of the heart in the 3D anatomical model can be modified on the basis of weight of the subject. The heart tends to be positioned more horizontally with increasing age. Therefore, a rotation of the heart in the 3D anatomical model can be modified on the basis of the age of the subject.

Thus, it is possible to provide a good approximation of a subject-specific 3D anatomical model, by obtaining an appropriate non-patient-specific 3D anatomical model, e.g. from the database, and modifying the non-patient-specific 3D anatomical model into a patient-specific 3D model of the heart and torso of the subject on the basis i.a. of the ultrasound data.

More in general is provided a method, such as a computer implemented method, for processing measurement data from electrocardiogram, ECG, electrodes on a subject. The method includes obtaining a three-dimensional, 3D, image of the torso of the subject including position information of the electrodes, e.g. using a 3D camera. The method includes obtaining, e.g. selecting from a database, a non-patient-specific 3D anatomical model of the heart and torso for the subject on the basis of the 3D image. Alternatively, or additionally, the non-patient-specific 3D anatomical model can e.g. be obtained from parameters derived from the 3D image. Alternatively, or additionally, the non-patient-specific 3D anatomical model can e.g. be obtained from a machine learning device on the basis of the 3D image. The method includes obtaining at least two, preferably two to four, two-dimensional, 2D, cross sectional images of the heart of the subject and modifying the non-patient-specific three-dimensional anatomical model into a patient-specific three-dimensional model of the heart and torso of the subject on the basis of the at least two cross sectional images. The cross sectional images can e.g. include ultrasound images, 2D X-ray images, or the like. The method includes determining a position of each electrode in the patient-specific three-dimensional anatomical model based on the three-dimensional image. The method includes obtaining electrocardiogram data from the electrodes and using the electrocardiogram data and the positions of the electrodes in the three-dimensional patient-specific anatomical model for estimating the distribution, fluctuation and/or movement of electrical activity through heart tissue.

According to an aspect is provided a, e.g. computer implemented, method for determining a patient-specific three-dimensional anatomical model of a heart and torso of a subject. The method includes obtaining a three-dimensional image of the torso of the subject. The method includes obtaining, e.g. from a database, a non-patient-specific three-dimensional anatomical model of the heart and torso for the subject on the basis of the three-dimensional image. The method includes obtaining ultrasound data of the heart of the subject and modifying the non-patient-specific three-dimensional anatomical model into a patient-specific three-dimensional model of the heart and torso of the subject on the basis of the ultrasound data.

More in general is provided a, e.g. computer implemented, method for determining a patient-specific three-dimensional anatomical model of a heart and torso of a subject. The method includes obtaining a three-dimensional image of the torso of the subject. The method includes obtaining, e.g. from a database, a non-patient-specific three-dimensional anatomical model of the heart and torso for the subject on the basis of the three-dimensional image. The method includes obtaining at least two, preferably two to four, two-dimensional, 2D, cross sectional images of the heart of the subject and modifying the non-patient-specific three-dimensional anatomical model into a patient-specific three-dimensional model of the heart and torso of the subject on the basis of the at least two cross sectional images. The cross sectional images can e.g. include ultrasound images, 2D X-ray images, or the like.

According to an aspect is provided a, e.g. computer implemented, method for determining a patient-specific three-dimensional anatomical model of an organ and torso of a subject. The method includes obtaining a three-dimensional image of the torso of the subject. The method includes obtaining, e.g. from a database, a non-patient-specific three-dimensional anatomical model of the organ and torso for the subject on the basis of the three-dimensional image. The method includes obtaining ultrasound data of the organ of the subject and modifying the non-patient-specific three-dimensional anatomical model into a patient-specific three-dimensional model of the organ and torso of the subject on the basis of the ultrasound data.

According to an aspect is provided a system for processing measurement data from electrocardiogram, ECG, electrodes on a subject. The system includes a processor, The processor is configured to obtain, from a three-dimensional camera, a three-dimensional image of the torso of the subject including position information of the electrodes. The processor is configured to obtain, e.g. from a database, a non-patient-specific three-dimensional anatomical model of the heart and torso for the subject on the basis of the three-dimensional image. The processor is configured to obtain, from one or more ultrasound probes, ultrasound data of the heart of the subject and modify the non-patient-specific three-dimensional anatomical model into a patient-specific three-dimensional model of the heart and torso of the subject on the basis of the ultrasound data. The processor is configured to determine a position of each electrode in the patient-specific three-dimensional anatomical model based on the three-dimensional image. The processor is configured to obtain electrocardiogram data from the electrodes; and use the electrocardiogram data and the positions of the electrodes in the three dimensional patient-specific anatomical model for estimating the distribution, fluctuation and/or movement of electrical activity through heart tissue.

Alternatively, or additionally, the processor is configured to obtain at least two, preferably two to four, two-dimensional, 2D, cross sectional images of the heart of the subject and to modify the non-patient-specific three-dimensional anatomical model into a patient-specific three-dimensional model of the heart and torso of the subject on the basis of the at least two cross sectional images. The cross sectional images can e.g. include 2D X-ray images, or the like.

Optionally, the processor is configured to determine a position and/or orientation of an ultrasound probe on the basis of the three-dimensional image, e.g. as explained hereinabove.

Optionally, the system includes a plurality of ultrasound probes. The one or more ultrasound probes can each include an ultrasound transceiver arranged for transmitting and receiving ultrasound signals.

Optionally, at least one of the electrodes includes an ultrasound probe. Thus one or more combined ECG/ultrasound probes are provided. The combined ECG ultrasound probes can include an ultrasound transceiver.

Optionally the system includes an ultrasound transmitter. The ultrasound transmitter can be separate from the ultrasound probes. The ultrasound probes can be ultrasound receivers. The ultrasound transmitter can e.g. be positioned on a back side of the subject, while one or more ultrasound receivers can be positioned on a front side of the subject. The ultrasound transmitter can be mounted in or on a table on which the subject lies during the measurement.

Optionally, the system includes a robot, such as a robot arm, wherein the processor is configured to cause the robot to position the electrodes and/or ultrasound probe(s) on the basis of a three-dimensional image of the torso of the subject.

According to an aspect the processor can cause an initial 3D image of the subject can be obtained. Then the processor can determine desired locations for the electrodes and/or ultrasound probes and cause the robot to position the electrodes and/or probes accordingly. The processor can then obtain the ECG data and ultrasound data. The processor can obtain a non-patient-specific three-dimensional anatomical model of the heart and torso for the subject. The processor can modify the non-patient-specific three-dimensional anatomical model into a patient-specific three-dimensional model of the heart and torso of the subject on the basis of the ultrasound data. The processor can determine a position of each electrode in the patient-specific three-dimensional anatomical model based on the determine desired locations for the electrodes. The processor can use the ECG data and the positions of the electrodes in the three-dimensional patient-specific anatomical model for estimating the distribution, fluctuation and/or movement of electrical activity through heart tissue.

Optionally, the processor is configured to synchronize the obtaining of the ultrasound data to a heart rhythm obtained from the electrocardiogram data obtained from the electrodes.

Optionally the processor is configured to determine whether or not the obtained ultrasound data is sufficient for modifying the non-patient-specific three-dimensional anatomical model into a patient-specific three-dimensional model of the heart and torso, and if the obtained ultrasound data is insufficient to obtain additional ultrasound data of the heart of the subject.

Optionally, the processor is configured to determine a desired position for the additional location and/or a desired orientation for the additional angle.

Optionally, the processor is configured for indicating the desired position and/or the desired orientation to a user.

Optionally, the processor is configured to cause the robot to position the ultrasound probe(s) to the desired position and/or the desired orientation.

Optionally, the processor is configured to estimate the position of heart scar tissue on the basis of absence or limited motion of the heart wall in the ultrasound data.

Optionally, the processor is configured to select the non-patient-specific three-dimensional anatomical model of the heart and torso on the basis of thorax contours determined from the three-dimensional image.

Optionally, the processor is configured to select a non-patient-specific three-dimensional anatomical model of the heart and torso on the basis of at least one of gender, age, weight, body length, chest circumference, frame size, and body-mass-index.

Optionally, the processor is configured to align the three-dimensional image and the non-patient-specific three-dimensional anatomical model.

Optionally, the processor is configured to scale the three-dimensional image to the obtained non-patient-specific three-dimensional anatomical model and/or scale the obtained non-patient-specific three-dimensional anatomical model to the three-dimensional image.

Optionally, the processor is configured to modify a position and/or orientation of the heart in the obtained non-patient-specific three-dimensional anatomical model on the basis of the ultrasound data.

According to an aspect is provided a non-transitory computer readable medium storing computer implementable instructions which when implemented by a programmable computer cause the computer to:
obtain a three-dimensional image of the torso of the subject including position information of the electrodes;
obtain a non-patient-specific three-dimensional anatomical model of the heart and torso for the subject on the basis of the three-dimensional image;
obtain ultrasound data of the heart of the subject;
modify the non-patient-specific three-dimensional anatomical model into a patient-specific three-dimensional model of the heart and torso of the subject on the basis of the ultrasound data;
determine a position of each electrode in the patient-specific three-dimensional anatomical model based on the three-dimensional image;
obtain electrocardiogram data; and
use the electrocardiogram data and the positions of the electrodes in the three dimensional patient-specific anatomical model for estimating the distribution, fluctuation and/or movement of electrical activity through heart tissue.

It will be appreciated that any of the aspects, features and options described in view of the ECG device or method apply equally to the other methods, systems and computer readable medium, and vice versa. It will also be clear that any one or more of the above aspects, features and options can be combined.

### BRIEF DESCRIPTION OF THE DRAWINGS

Embodiments of the present invention will now be described in detail with reference to the accompanying drawings in which:

FIGS. 1A, 1B, 1C and 1D show an example of a system;

FIG. 2 shows an example of a cross section of a torso;

FIGS. 3A, 3B, 3C and 3D show an example of a system;

FIG. 4 shows an example of a cross section of a torso;

FIG. 5 shows an example of a method;

FIG. 6 shows an example of part of a method;

FIG. 7 shows an example of part of a method;

FIG. 8 shows an example of part of a method;

FIG. 9 shows an example of part of a method;

FIG. 10 shows an example of part of a method;

FIG. 11 shows an example of part of a method; and

FIG. 12 shows an example of part of a method.

### DETAILED DESCRIPTION

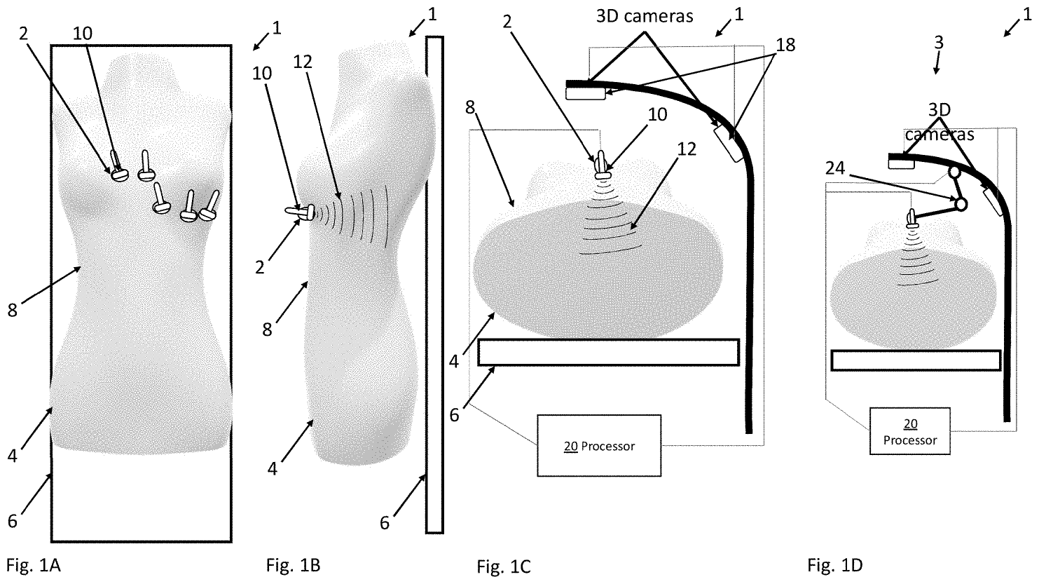

FIGS. 1A, 1B and 1C show an example of a system 1 for processing measurement data from electrocardiogram, ECG, electrodes 2 on a subject 4. FIG. 1A shows a top view, FIG. 1B a side view, and FIG. 1C a cross sectional view. FIGS. 1A-1C show a table 6 on which the subject 4 is positioned. On the torso 8 of the subject 4 a plurality of ECG electrodes 2 is placed. In this example a plurality of ultrasound probes 10 is placed on the torso 8. Here, each electrode 2 is provided with a probe 10. Hence, a plurality of ECG electrode/ultrasound probes is provided. The electrodes 2 are arranged to detect surface potentials on the torso 8. The ultrasound probes 10 are arranged to emit an ultrasound signal 12 into the torso 8 and receive a reflected ultrasound signal 14 from the torso.

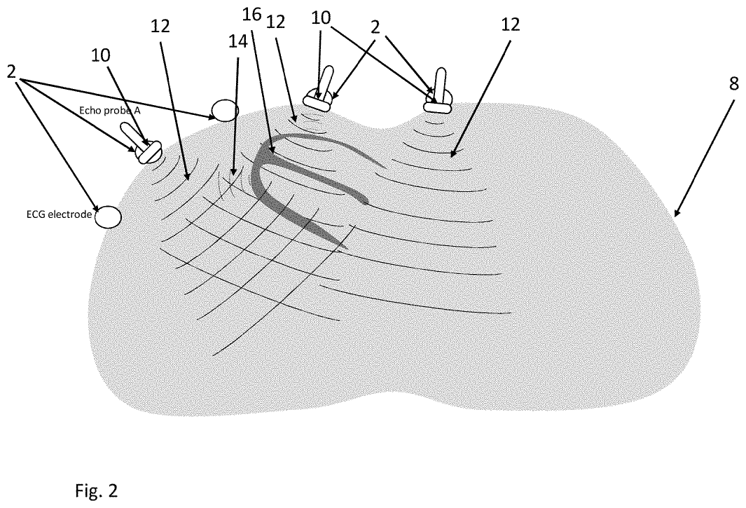

FIG. 2 shows an example of a cross section of the torso 8. In this example the heart 16 is indicated. The ultrasound signal 12 transmitted into the torso 8 by the probe 10 and the reflected ultrasound signal 14 reflected from the heart 16 towards the probe 10 are also indicated in FIG. 2. Note that in FIG. 2 not every ECG electrode 2 is provided with an ultrasound probe 10.

Returning to FIG. 1C the system 1 is provided with at least one 3D camera 18, here two 3D cameras. The system further comprises a processor 20. Here, the processor 20 is communicatively connected to the 3D camera 20, the electrodes 2 and the probes 10.

In this example, the electrodes 2, ultrasound probes 10, 3D cameras 18 and processor 20 form an electrocardiogram, ECG, device 3. The ECG device 3 is arranged for performing inverse electrocardiography as will be explained below.

The electrodes 2, ultrasound probes 10, or combined electrodes/probes can be attached to the subject 4 e.g. by an adhesive. It is also possible that the system includes a robot 24, such as a robot arm, controlled by the processor 20 for positioning the electrodes, probes or electrodes/probes onto the subject, as shown in FIG. 1D.

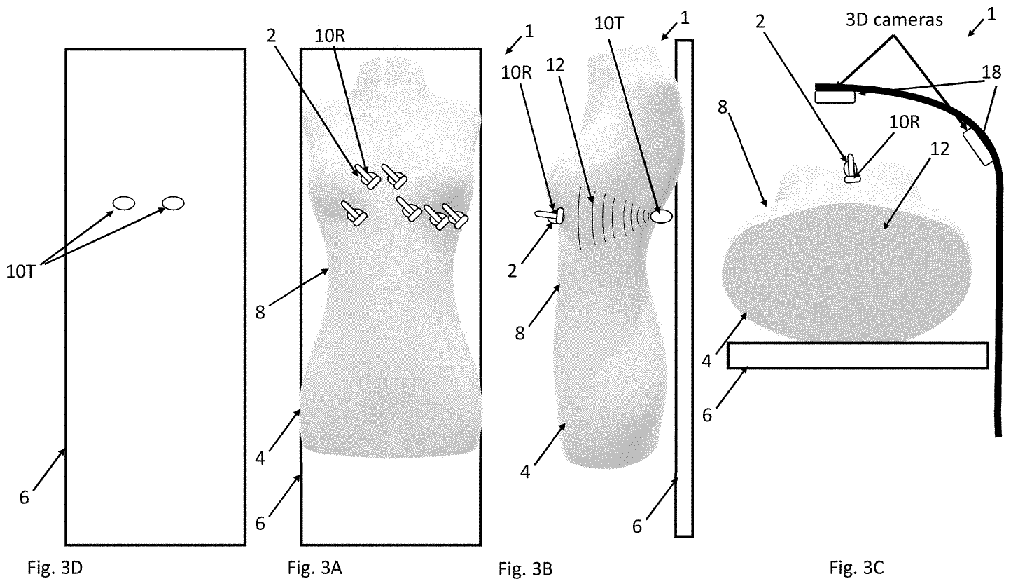

FIGS. 3A, 3B, 3C and 3D show an alternate example of a system 1 for processing measurement data from electrocardiogram, ECG, electrodes 2 on a subject 4. The system of FIGS. 3A-3D is similar to that shown in FIGS. 1A-1C, with the difference that in this example the table 6 includes one or more ultrasound transmitters 10T. In this example, the ultrasound probes are ultrasound receivers 10R. Similar to FIG. 1D a robot 24 may be used to position the receivers 10R.

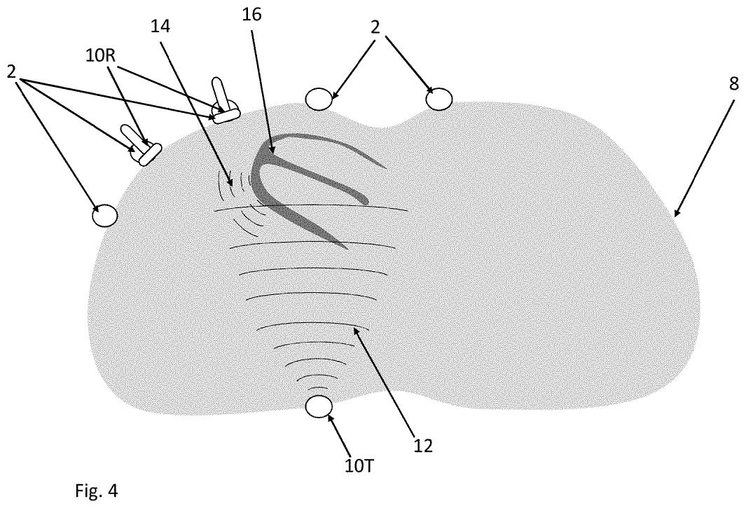

FIG. 4 shows an example of a cross section of the torso 8. In this example the heart 16 is indicated. The ultrasound signal 12 transmitted into the torso 8 by the transmitter 10T and the reflected ultrasound signal 14 reflected from the heart 16 towards the receiver 10R are also indicated in FIG. 4. Note that in FIG. 4 not every ECG electrode 2 is provided with an ultrasound probe 10.

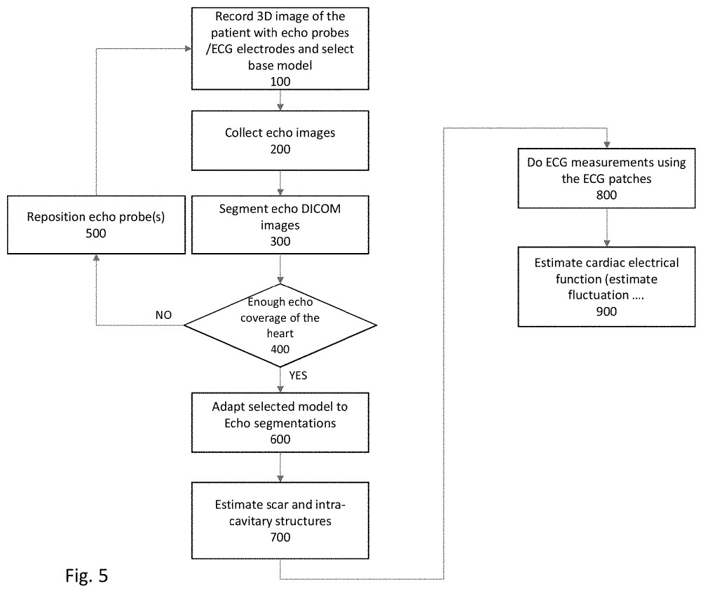

The system 1, and the ECG device 3, as described can be used as follows, referring to the exemplary method 99 of FIG. 5. The processor 20 obtains in step 100 a three-dimensional image of the torso 8 of the subject 4 and selects, from a database 22, a non-patient-specific 3D anatomical model of the heart and torso for the subject. Thereto the processor 20 can control the 3D camera(s) 18 to obtain the 3D image. From the ultrasound probes 10 the processor 20 obtains in step 200 ultrasound data of the heart 16 of the subject 4. On the basis of the ultrasound data, in step 600 the processor 20 modifies the non-patient-specific 3D anatomical model into a patient-specific 3D model of the heart 16 and torso 8 of the subject 4. From the 3D image the processor 20 determines a position of each electrode 2 on the torso 4 and relates this to a position in the patient-specific 3D anatomical model. From the electrodes 2 the processor 20 obtains ECG data in step 800. Finally, using the ECG data and the positions of the electrodes in the 3D patient-specific anatomical model the processor 20 determines a 3D model of electrical heart activity for the subject in step 900. The processor can e.g. estimate the distribution, fluctuation and/or movement of electrical activity through heart tissue.

Hence, the system 1, and the ECG device 3, is arranged to perform inverse electrocardiography. The system 1 or the ECG device 3 can include display means, such as a display screen or printer, for displaying the 3D model of electrical heart activity to a user. The 3D model of electrical heart activity can be displayed as a, e.g. rotatable and/or movable and or scalable, 2D rendering on the display means. The 3D model of electrical heart activity can e.g. have a value representative of the electrical heart activity associated with each location, such as each node, on the surface of the 3D model of the heart. The values can e.g. represent electrical activation sequence, distribution, fluctuation and/or movement of electrical activity through heart tissue, heart synchronicity, or the like. The 3D model of electrical heart activity can be represented in false colors on the surface of the 3D model of the heart. Each value can e.g. be associated with a particular color. The 3D model of electrical heart activity can e.g. be represented in contours of equal value on the surface of the patient-specific 3D model of the heart.

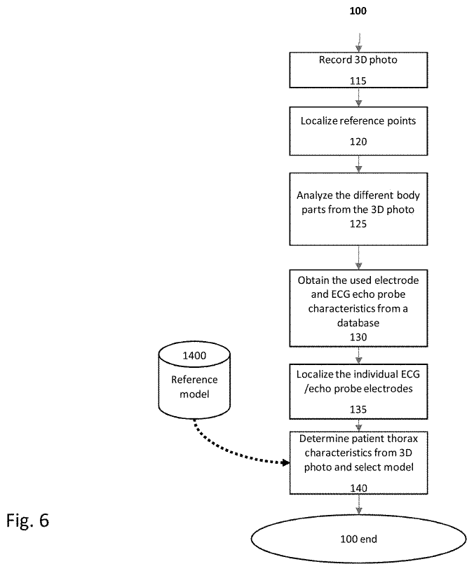

FIG. 6 shows an exemplary elaboration of step 100. In step 115 the processor 20 obtains the 3D image. In this example, in step 120 the processor 120 localizes reference points in the 3D image. Such reference points can e.g. be anatomical reference points, such as shoulders, xyphoid, etc. The reference points can also be artificial reference points such as markers (e.g. stickers) applied to the torso 4. In step 125 the processor 20 analyzes different body parts from the 3D image. The different body parts can be investigated to determine a local normal vector orthogonal to the thorax surface. A significant amount of adipose tissue can enable the operator to position the ultrasound probe in the optimal direction, ribs may do the opposite. The presence of adipose tissue and/or ribs can be deduced from the 3D image. The step 130 the processor 20 obtains the used electrode 2 and ultrasound probe 10 characteristics from a database. An ultrasound beam generally points away from the ultrasound probe in a fan shaped pattern. The shape and/or direction of the fan relative to the ultrasound probe may vary with the type of probe. The processor 30 can e.g. retrieve ultrasound fan shape and/or direction relative to the ultrasound probe from the database. In step 135 the processor localizes the individual electrode 2/ultrasound probes 10 in the 3D image. In step 140 the processor determines subject thorax characteristics from the 3D image and selects, from a database 1400, a non-patient-specific 3D model based on the thorax characteristics.

The selection of the non-patient-specific 3D anatomical model is here based on the 3D image, e.g. on thorax contours determined from the 3D image. The selecting here includes selecting, from the plurality of non-patient-specific 3D anatomical models in the database, the 3D anatomical model showing closest conformity to the torso of the subject. The selection may be based on visual comparison of the 3D image of the torso of the subject with the 3D models in the database. Such selection may be automated on the basis of pattern recognition. The selection may, e.g. additionally, be made on the basis of parameters, such as gender, age, weight, body length, chest circumference, frame size, BMI, etc. of the subject 4.

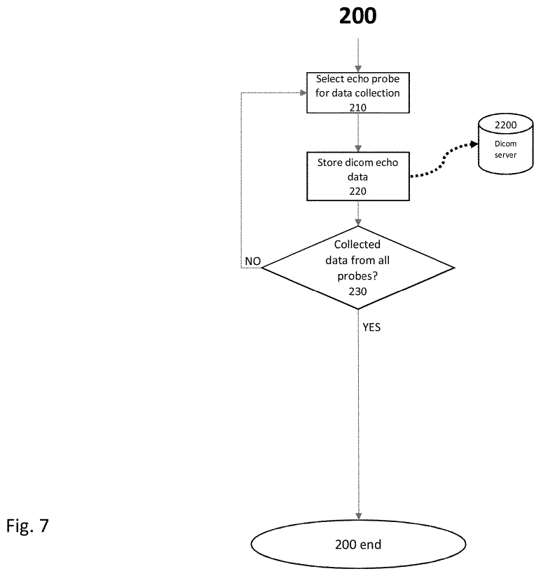

FIG. 7 shows an exemplary elaboration of step 200. In step 210 the processor 20 selects an ultrasound probe for collecting ultrasound data, i.e. an ultrasound image obtained by that respective probe 10. Tin this example, the ultrasound data is stored at a DICOM (Digital Imaging and Communications in Medicine) server 2200 in step 220. In step 230 the processor 20 checks whether ultrasound data has been collected from all ultrasound probes 10. If not, ultrasound data of the next probe is collected. It will be appreciated that it is not absolutely necessary that ultrasound data is collected from all ultrasound probes. In some cases the ultrasound data from a sub-set of the ultrasound probes may suffice.

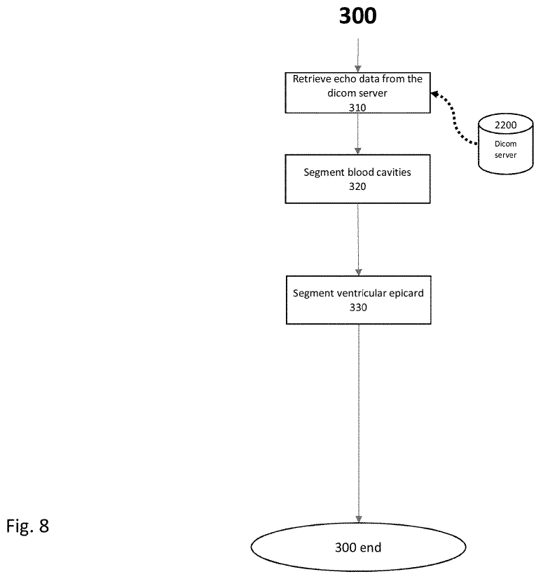

It is possible that the method 99 includes the step 300 of segmenting the ultrasound DICOM images. The goal of segmenting is to simplify and/or change the representation of the DICOM images into something that is more meaningful and easier to analyze. Image segmenting is typically used to locate objects and boundaries in images, such as the heart or parts of the heart in the ultrasound images. When applied to a plurality of ultrasound images the resulting points and/or contours after image segmenting can be used to create a 3D reconstruction of the heart, e.g. with the help of interpolation algorithms. Preferably the ultrasound images segmenting is performed at the same trigger time for each ultrasound image, e.g. all images at diastolic cardiac phase. FIG. 8 shows an exemplary elaboration of step 300. In step 310 the ultrasound data is retrieved from the DICOM server 2200. In step 320 blood cavities in the ultrasound images are segmented. In step 330 the ventricular epicard is segmented.

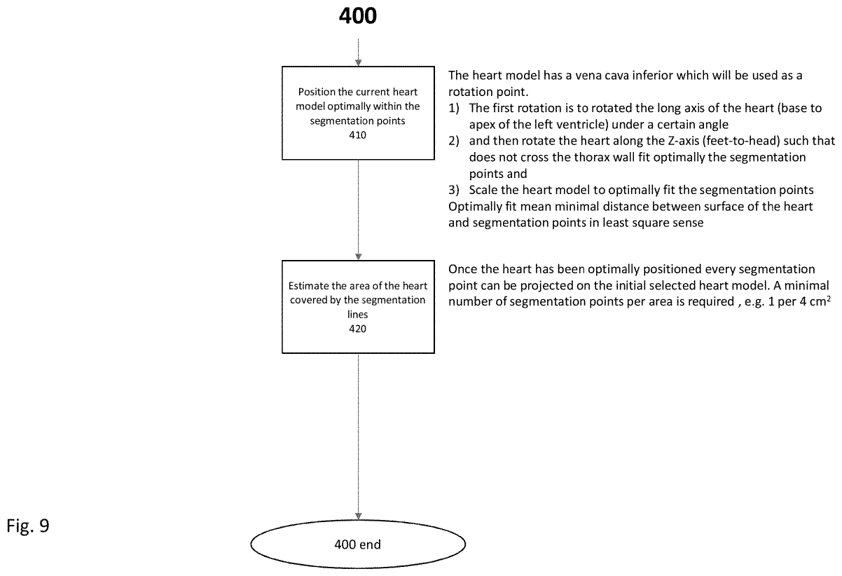

It is possible that the method 99 includes the step 400 of determining whether enough ultrasound data has been collected, e.g. a large part of the heart surface has been captured. For instance when just an endocardium is captured, and only a part of the epicardium, a constant wall thickness can be assumed to estimate the epicardial surface based on the endocardial segmentation points. FIG. 9 shows an exemplary elaboration of step 400. In step 410 a position of the current heart model, i.e. the heart model of the non-patient-specific 3D anatomical model, is modified to optimally correspond with the segmentation points. In an example, the current heart model has a vena cava inferior which is used as a rotation point. Repositioning of the heart can include in a first step rotating the current heart model about the long axis (base to apex of the left ventricle) of the heart. Repositioning of the heart can include in a second step rotating the current heart model about the Z-axis (feet-to-head) such that the heart model does not intersect the thorax wall and fits optimally with the segmentation points. Repositioning of the heart can include in a third step scaling the current heart model to optimally fit the segmentation points. Herein “optimally fit” in this example means a minimal distance between the surface of the heart model and the segmentation points in a least square sense.

In step 420 an area of the heart model covered by segmentation contours is estimated. Once the heart model has been optimally positioned every segmentation point can be projected on the initial selected heart model. A minimal number of segmentation points per area is required, e.g. 1 per 4 cm2. If in step 400 it is determined that ultrasound data coverage of the heart is insufficient, in step 500 one or more ultrasound probes 10 can be repositioned and additional ultrasound data can be obtained from the repositioned probe(s) 10. In an example, the system 1 can include a robot 24. The processor 20 can be configured to control the robot 24 to reposition one or more of the ultrasound probes 10.

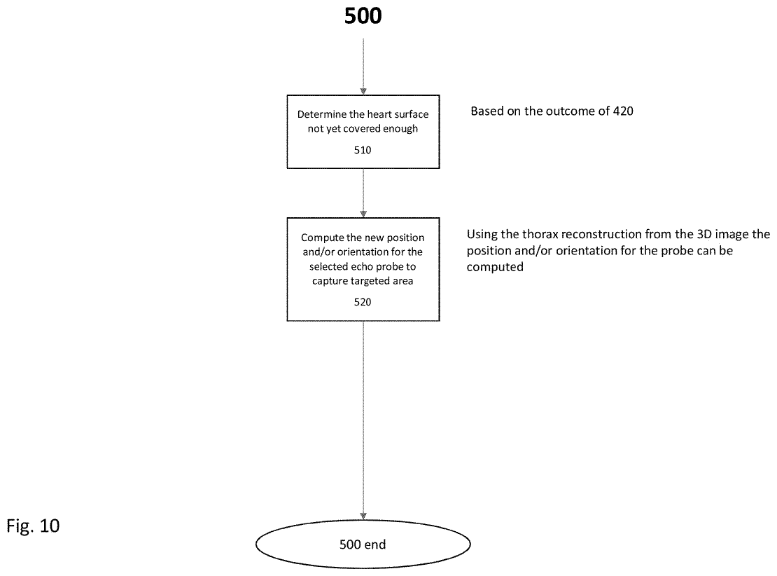

FIG. 10 shows an exemplary elaboration of step 500. In step 510 the processor 20 determines the area of the heart surface not sufficiently covered by ultrasound data, e.g. based on the outcome of step 420. In step 520 a new position and/or orientation for a selected one or more ultrasound probes 10 is determined for obtaining an ultrasound image of the area of the heart surface not yet sufficiently covered by ultrasound data.

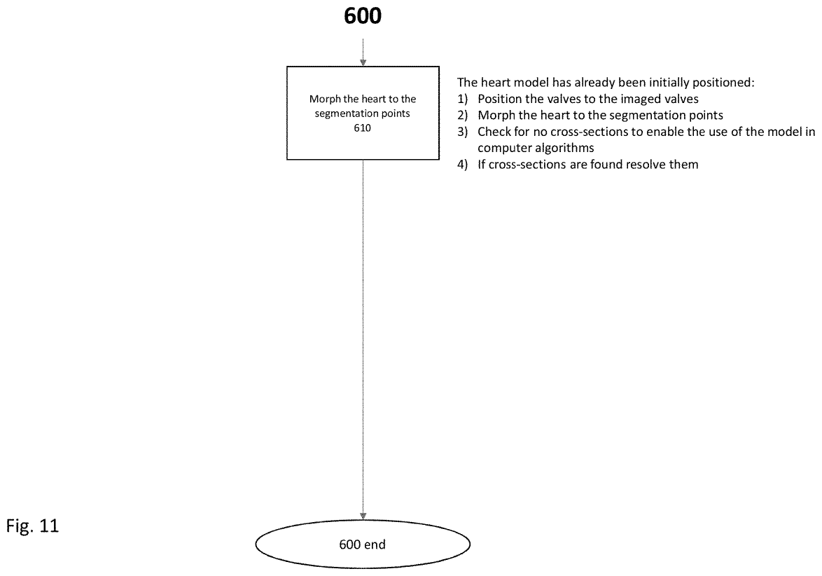

FIG. 11 shows an exemplary elaboration of step 600. In step 610 the heart model can be morphed to the segmentation points. The heart model can already be repositioned in step 410. In step 610 the valves of the heart model can be repositioned to correspond to the position of the imaged valves. The heart can be morphed to the segmentation points. The modified heart model can be checked to be free from areas where the model intersects itself. If model intersections are detected these are resolved. Hence, the intersection-free model can easily be used in computer algorithms.

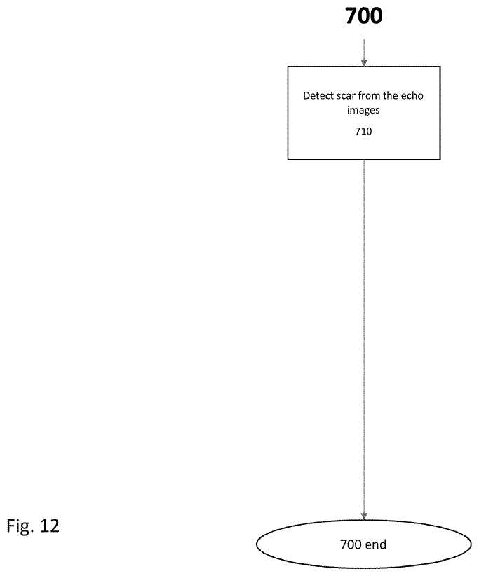

FIG. 12 shows an exemplary elaboration of step 700. In step 710 scar tissue is detected from the ultrasound images. For instance, the ultrasound data can be obtained during the cardiac cycle to determine locations on the heart wall that do not move, or at least move significantly less than to be expected in a healthy heart. Such locations can be used as indications of scar tissue presence.

Herein, the invention is described with reference to specific examples of embodiments of the invention. It will, however, be evident that various modifications and changes may be made therein, without departing from the essence of the invention. For the purpose of clarity and a concise description features are described herein as part of the same or separate embodiments, however, alternative embodiments having combinations of all or some of the features described in these separate embodiments are also envisaged.

In the examples the method includes selecting from a database, a non-patient-specific 3D anatomical model of the heart and torso for the subject on the basis of the 3D image. Alternatively, or additionally, the non-patient-specific 3D anatomical model can e.g. be obtained from parameters derived from the 3D image. Alternatively, or additionally, the non-patient-specific 3D anatomical model can e.g. be obtained from a machine learning device on the basis of the 3D image.

However, other modifications, variations, and alternatives are also possible. The specifications, drawings and examples are, accordingly, to be regarded in an illustrative sense rather than in a restrictive sense.

For the purpose of clarity and a concise description features are described herein as part of the same or separate embodiments, however, it will be appreciated that the scope of the invention may include embodiments having combinations of all or some of the features described.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

1. An electrocardiogram, ECG, device comprising:
one or more electrodes arranged to be placed on a subject;
one or more ultrasound probes;
a three-dimensional camera; and
a processor configured to:
obtain, from the three-dimensional camera, a three-dimensional image of the torso of the subject including position information of the electrodes;
obtain, e.g. from a database, a non-patient-specific three-dimensional anatomical model of the heart and torso for the subject on the basis of the three-dimensional image;
obtain, from the one or more ultrasound probes, ultrasound data of the heart of the subject;
modify the non-patient-specific three-dimensional anatomical model into a patient-specific three-dimensional model of the heart and torso of the subject on the basis of the ultrasound data;
determine a position of each electrode in the patient-specific three-dimensional anatomical model based on the three-dimensional image;
obtain electrocardiogram data from the electrodes; and
use the electrocardiogram data and the positions of the electrodes in the three dimensional patient-specific anatomical model for determining a three-dimensional model of electrical heart activity.

2. The ECG device of claim 1, wherein the processor is configured to determine a position and/or orientation of an ultrasound probe on the basis of the three-dimensional image.

3. The ECG device of claim 1, wherein at least one of the electrodes includes an ultrasound probe.

4. The ECG device of claim 1, including an ultrasound transmitter.

5. The ECG device of claim 1, including a robot, wherein the processor is configured to cause the robot to position the electrode(s) and/or ultrasound probe(s) on the basis of a three-dimensional image of the torso of the subject.

6. The ECG device of claim 1, wherein the processor is configured to synchronize the obtaining of the ultrasound data to a heart rhythm obtained from the electrocardiogram data obtained from the electrodes.

7. The ECG device of claim 1, wherein the processor is configured to determine whether or not the obtained ultrasound data is sufficient for modifying the non-patient-specific three-dimensional anatomical model into a patient-specific three-dimensional model of the heart and torso, and if the obtained ultrasound data is insufficient to proceed to obtain additional ultrasound data of the heart of the subject.

8. The ECG device of claim 1, including display means for displaying the three-dimensional model of electrical heart activity to a user.

9-25. (canceled)

26. A system for processing measurement data from electrocardiogram, ECG, electrodes on a subject, the system including a processor configured to:
obtain, from a three-dimensional camera, a three-dimensional image of the torso of the subject including position information of the electrodes;
obtain, e.g. from a database, a non-patient-specific three-dimensional anatomical model of the heart and torso for the subject on the basis of the three-dimensional image;
obtain, from one or more ultrasound probes, ultrasound data of the heart of the subject;
modify the non-patient-specific three-dimensional anatomical model into a patient-specific three-dimensional model of the heart and torso of the subject on the basis of the ultrasound data;
determine a position of each electrode in the patient-specific three-dimensional anatomical model based on the three-dimensional image;
obtain electrocardiogram data from the electrodes; and
use the electrocardiogram data and the positions of the electrodes in the three dimensional patient-specific anatomical model for estimating the distribution, fluctuation and/or movement of electrical activity through heart tissue.

27. The system of claim 26, wherein the processor is configured to determine a position and/or orientation of an ultrasound probe on the basis of the three-dimensional image.

28. The system of claim 26, including a plurality of ultrasound probes.

29. The system of claim 26, wherein at least one of the electrodes includes an ultrasound probe.

30. The system of claim 26, including an ultrasound transmitter.

31. The system of claim 26, including a robot, wherein the processor is configured to cause the robot to position the electrodes and/or ultrasound probe(s) on the basis of a three-dimensional image of the torso of the subject.

32. The system of claim 26, wherein the processor is configured to synchronize the obtaining of the ultrasound data to a heart rhythm obtained from the electrocardiogram data obtained from the electrodes.

33. The system of claim 26, wherein the processor is configured to determine whether or not the obtained ultrasound data is sufficient for modifying the non-patient-specific three-dimensional anatomical model into a patient-specific three-dimensional model of the heart and torso, and if the obtained ultrasound data is insufficient to proceed to obtain additional ultrasound data of the heart of the subject.

34. The system of claim 33, wherein the processor is configured to determine a desired position for the additional location and/or a desired orientation for the additional angle.

35. The system of claim 34, wherein the processor is configured for indicating the desired position and/or the desired orientation to a user.

36. The system of claim 34, wherein the processor is configured to cause the robot to position the ultrasound probe(s) to the desired position and/or the desired orientation.

37. The system of claim 26, wherein the processor is configured to estimate the position of heart scar tissue on the basis of absence or limited motion of the heart wall in the ultrasound data.

38. The system of claim 26, wherein the processor is configured to select the non-patient-specific three-dimensional anatomical model of the heart and torso on the basis of thorax contours determined from the three-dimensional image.

39. The system of claim 26, wherein the processor is configured to select a non-patient-specific three-dimensional anatomical model of the heart and torso on the basis of at least one of gender, age, weight, body length, chest circumference, frame size, and body-mass-index.

40. The system of claim 26, wherein the processor is configured to align the three-dimensional image and the non-patient-specific three-dimensional anatomical model.

41. The system of claim 26, wherein the processor is configured to scale the three-dimensional image to the obtained non-patient-specific three-dimensional anatomical model and/or scale the obtained non-patient-specific three-dimensional anatomical model to the three-dimensional image.

42. The system of claim 26, wherein the processor is configured to modify a position and/or orientation of the heart in the obtained non-patient-specific three-dimensional anatomical model on the basis of the ultrasound data.

43. A non-transitory computer readable medium storing computer implementable instructions which when implemented by a programmable computer cause the computer to:
obtain a three-dimensional image of the torso of the subject including position information of the electrodes;
obtain a non-patient-specific three-dimensional anatomical model of the heart and torso for the subject on the basis of the three-dimensional image;
obtain ultrasound data of the heart of the subject;
modify the non-patient-specific three-dimensional anatomical model into a patient-specific three-dimensional model of the heart and torso of the subject on the basis of the ultrasound data;
determine a position of each electrode in the patient-specific three-dimensional anatomical model based on the three-dimensional image;
obtain electrocardiogram data; and
use the electrocardiogram data and the positions of the electrodes in the three dimensional patient-specific anatomical model for estimating the distribution, fluctuation and/or movement of electrical activity through heart tissue.
